(19) United States
(12) Patent Application Publication
(10) Pub. No.: US 2025/0275902 A1
(43) Pub. Date: Sep. 4, 2025

(54) COMPOSITION COMPRISING BIOSURFACTANT AND SALICYLIC ACID

(71) Applicant: L'OREAL, Paris (FR)

(72) Inventors: Christopher PANG, New York, NY (US); Changlong Chen, Edison, NJ (US); Siliu Tan, Westfield, NJ (US); Mariko Hasebe, New York, NY (US); Hiroaki Kaga, Kawasaki (JP); Masanori Orita, Kawasaki (JP)

(73) Assignee: L'OREAL, Paris (FR)

(21) Appl. No.: 19/067,315

(22) Filed: Feb. 28, 2025

Related U.S. Application Data
(63) Continuation of application No. 18/901,492, filed on Sep. 30, 2024.
(63) Continuation of application No. 18/591,178, filed on Feb. 29, 2024.
(63) Continuation of application No. 18/591,930, filed on Feb. 29, 2024.

(30) Foreign Application Priority Data
Apr. 30, 2024 (FR) 2404520
Jun. 10, 2024 (FR) 2406067
Nov. 21, 2024 (FR) 2412751

(51) Int. Cl. A61K 8/365; A61K 8/02; A61K 8/60; A61Q 19/10

(52) U.S. Cl. CPC A61K 8/365; A61K 8/0212; A61K 8/602; A61Q 19/10

(57) ABSTRACT

The present invention relates to compositions comprising (a) at least one glycolipid; (b) at least one salicylic acid or its derivatives represented by formula (I); (c) at least one cationic polymer which may be a polysaccharide derived from β-linked D-glucosamine and N-acetyl-D-glucosamine; (d) at least one clay; and (e) cosmetically acceptable solvents comprising water, glycerin. The compositions according to the present invention can provide an effect of treating, hydrating, and/or cleansing acne-prone skin.

### FIELD OF THE INVENTION

The present invention relates to compositions comprising at least one biosurfactant and at least one salicylic acid or its derivatives, in particular a cosmetic composition comprising the same.

### CROSS-REFERENCE TO RELATED APPLICATIONS

This application claims benefit of and priority to U.S. Non-Provisional application Ser. No. 18/901,492, filed Sep. 30, 2024, which claims benefit of and priority to U.S. Non-Provisional application Ser. No. 18/591,930, filed Feb. 29, 2024, U.S. Non-Provisional application Ser. No. 18/591,930, filed Feb. 29, 2024, French Patent Application No. FR 2404520, filed Apr. 30, 2024, and French Patent Application No. FR 2406067, filed Jun. 10, 2024, and this application also claims benefit of and priority to U.S. Non-Provisional application Ser. No. 18/591,178, filed Feb. 29, 2024, and U.S. Non-Provisional application Ser. No. 18/591,930, filed Feb. 29, 2024, and each of French Patent Application No. FR 2404520, filed Apr. 30, 2024, French Patent Application No. FR 2406067, filed Jun. 10, 2024, and French Patent Application No. FR 2412751, filed Nov. 21, 2024, each of which is incorporated herein by reference in its entirety.

### BACKGROUND OF THE INVENTION

Anti-acne cleansing compositions often have the undesired effect of drying out the skin, and are further limited in the scope of their utility for purposes of use and ease of application for cleansing and extended applications as a mask or spot treatment. Therefore, there is a need to develop an effective anti-acne composition which provides not only effectiveness for treating acne but also sensorial and aesthetic benefits and adequate hydration to acne prone-skin in a format that satisfies consumer preferences for application and use.

### BRIEF SUMMARY OF THE INVENTION

An objective of the present invention is to provide a composition for effectively treating and reducing acne, hydrating and/or cleansing acne-prone skin in a format that satisfies consumer preferences for application and use.

In some embodiments, the disclosure provides a composition that may have a unique paste texture, such as, but not limited to, a wasabi-like paste texture wherein the texture is sufficiently thick to not run or slip off the skin when applied prior to rinsing, is not slimy, and is smooth without granulation or graininess, is water miscible and glides on when applied with shear stress, with low to high foaming and which dries clear. As used herein, “unique wasabi-like paste” refers by comparison to, for example a familiar condiment known as wasabi paste made from horseradish, and may include one or more of water or other liquid, mustard, green food dye and other ingredients, wherein the inventive composition is not fibrous or grainy as may be the case with actual wasabi type condiments. The wasabi-like paste may be used as a 3-in-1 composition for acne-prone skin, which includes a daily skin clearing cleanser to be mixed with water, an overnight drying spot treatment in its original form, and/or a mask when layered on damped face. In some such embodiments, the composition includes a glycolipid selected from the group consisting of rhamnolipids, sophorolipids, glucolipids, trehalolipids, cellobiose lipids and mixtures thereof, at least one salicylic acid or derivative, at least on cationic polymer comprising a polysaccharide derived from β-linked D-glucosamine and N-acetyl-D-glucosamine, at least one clay, cosmetically acceptable solvents, at least one surfactant, at least one thickener/rheology modifier, one or more additional skin actives, and at least one thickener/rheology modifier. In some such embodiments, the composition includes a glycolipid comprising at least one rhamnolipid, at least one salicylic acid or derivative comprising salicylic acid, at least on cationic polymer comprising chitosan, at least one clay comprising kaolin, cosmetically acceptable solvents comprising at least water and glycerin, at least one surfactant that is a solid surfactant comprising sodium cocoyl isethionate, and at least on additional skin active comprising niacinamide, and at least one thickener/rheology modifier that includes carrageenan. In one such particular embodiment, the disclosure provides a composition that comprises the at least one rhamnolipid present from 2% to 5%, salicylic acid or its derivatives present from 1% to 3%, kaolin present from about 10% to about 20%, the at least one surfactant comprising sodium cocoyl isethionate present from about 5% to about 15%, glycerin present from 10% to 40%, niacinamide present at about 2%, carrageenan present from about from about 0.5% to about 2%, all amounts by weight, relative to the total weight of the composition, and wherein the pH of the composition is from about 3.5 to about 6.5.

The above objective of the present invention can be achieved by a composition comprising:
(a) at least one glycolipid:
(b) at least one salicylic acid or its derivatives of formula (I):

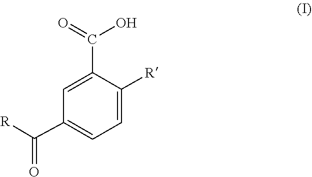

in which:
   the radical R denotes a linear, branched or cyclic, saturated aliphatic chain containing from 2 to 22 carbon atoms: an unsaturated chain containing from 2 to 22 carbon atoms containing one or more double bonds that may be conjugated; an aromatic nucleus linked to the carbonyl radical directly or via saturated or unsaturated aliphatic chains containing from 2 to 7 carbon atoms: said groups possibly being substituted with one or more substituents, which may be identical or different, chosen from halogen atoms, a trifluoromethyl group, hydroxyl groups in free form or esterified with an acid containing from 1 to 6 carbon atoms, or carboxyl groups in free form or esterified with a lower alcohol containing from 1 to 6 carbon atoms;
   R′ is a hydroxyl group or an ester group of formula:

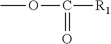

in which R₁ denotes a linear or branched, saturated or unsaturated aliphatic chain containing 1 to 18 carbon atoms; and also salts thereof derived from an inorganic or organic base;
(c) at least one cationic polymer selected from a polysaccharide derived from β-linked D-glucosamine or N-acetyl-D-glucosamine;
(d) at least one clay present from about 5% to about 25% by weight, relative to the total weight of the composition;
(e) cosmetically acceptable solvents comprising water, and comprising glycerin present from about 2% to about 40% by weight, relative to the total weight of the composition; and
(f) at least one surfactant present from about 1% to about 20% by weight, relative to the total weight of the composition,
wherein the pH of the composition is from about 3.5 to about 6.5, and
wherein the composition has a wasabi-like paste texture.

The above objective of the present invention can also be achieved by a composition comprising:
(a) at least one rhamnolipid present from 2% by weight to 5% by weight, relative to the total weight of the composition;
(b) at least one salicylic acid or its derivatives or its derivatives of formula (I):

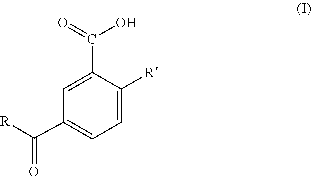

in which:
   the radical R denotes a linear, branched or cyclic, saturated aliphatic chain containing from 2 to 22 carbon atoms; an unsaturated chain containing from 2 to 22 carbon atoms containing one or more double bonds that may be conjugated; an aromatic nucleus linked to the carbonyl radical directly or via saturated or unsaturated aliphatic chains containing from 2 to 7 carbon atoms; said groups possibly being substituted with one or more substituents, which may be identical or different, chosen from halogen atoms, a trifluoromethyl group, hydroxyl groups in free form or esterified with an acid containing from 1 to 6 carbon atoms, or carboxyl groups in free form or esterified with a lower alcohol containing from 1 to 6 carbon atoms;
   R′ is a hydroxyl group or an ester group of formula:

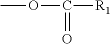

in which R1 denotes a linear or branched, saturated or unsaturated aliphatic chain containing 1 to 18 carbon atoms; and also salts thereof derived from an inorganic or organic base;
the at least one salicylic acid or its derivatives or its derivatives of formula (I) being present from about 1% by weight to about 3% by weight, relative to the total weight of the composition;
(c) at least one cationic polymer comprising chitosan;
(d) at least one clay present from about 5% to about 25% by weight, relative to the total weight of the composition:
(e) cosmetically acceptable solvents comprising water, and comprising glycerin present from about 10% to about 40% by weight, relative to the total weight of the composition; and
(f) at least one surfactant present from about 1% to about 20% by weight, relative to the total weight of the composition,
wherein the pH of the composition is from about 3.5 to about 6.5,
wherein the composition has a wasabi-like paste texture, and
wherein the composition is a 3-in-1 paste formula base for acne-prone skin as a skin cleanser, as a spot treatment, and as a mask.

The glycolipid is selected from the group consisting of rhamnolipids, sophorolipids, glucolipids, trehalolipids, cellobiose lipids and mixtures thereof. In one embodiment, the glycolipid is rhamnolipids.

In one embodiment, the at least one salicylic acid or its derivatives is salicylic acid.

The salicylic acid derivatives are represented by formula (I) in which the radical R denotes a linear, branched or cyclic, saturated aliphatic chain containing from 2 to 22 carbon atoms.

The salicylic acid derivatives are represented by formula (I) in which the R radical denotes a linear or branched alkyl or alkenyl group, alternatively alkyl group, containing 2 or more, alternatively 3 or more, alternatively 4 or more, alternatively 5 or more, alternatively 6 or more carbon atoms, and/or 22 or less, alternatively 18 or less, alternatively 14 or less, alternatively 12 or less, alternatively 10 or less carbon atoms.

The salicylic acid derivatives are represented by formula (I) in which R′ denotes a hydroxyl group.

The cationic polymer is a polysaccharide derived from β-linked D-glucosamine and N-acetyl-D-glucosamine, such as, but not limited to, chitosan.

The at least one clay, can be, such as, but not limited to, kaolin.

The cosmetically acceptable solvents comprise water, and comprise glycerin present from about 2% to about 40% by weight, relative to the total weight of the composition: or the cosmetically acceptable solvents comprise water, and comprise glycerin present from about 10% to about 30% by weight, relative to the total weight of the composition.

The compositions according to the present invention may further comprise at least one surfactant comprising sodium cocoyl isethionate present at about 10% by weight, relative to the total weight of the composition.

The compositions according to the present invention may also further comprise niacinamide present at about 2% by weight, relative to the total weight of the composition.

The pH of the composition is from about 3.5 to about 6.5.

The amount of the glycolipid(s), including rhamnolipids, may be 0.1% to 10% by weight, alternatively 1% to 7%, by weight, alternatively 2% to 5% by weight, alternatively 3% by weight, relative to the total weight of the composition.

The amount of the salicylic acid or its derivatives may be from 0.1% to 5% by weight, alternatively from 1% to 3% by weight, alternatively 2% by weight, relative to the total weight of the composition.

The amount of the clay may be from 5% to 25% by weight, alternatively from 10% to 20% % by weight, alternatively 15% by weight, relative to the total weight of the composition.

The amount of the glycerin may be from 1% to 45% by weight, alternatively from 2% to 40% by weight, alternatively 10% to 30% by weight, relative to the total weight of the composition.

The pH of the compositions according to the present invention is from about 3.5 to about 6.5, alternatively from about 4 to 6, alternatively from about 4.5 to 5.5, alternatively the pH is 5.5 or 5.0.

The at least one clay may include kaolin present from about 10% to about 20% by weight, relative to the total weight of the composition and the at least one surfactant may include sodium cocoyl isethionate present from about 5% to about 15% by weight, relative to the total weight of the composition. The kaolin may be present at about 15% by weight, relative to the total weight of the composition and the sodium cocoyl isethionate may be present at about 10% by weight, relative to the total weight of the composition.

The compositions of the present invention may have a paste texture, such as, but not limited to, a unique wasabi-like paste texture wherein the texture is sufficiently thick to not run or slip off the skin, is not slimy, and is smooth without granulation or graininess, is water miscible and glides on when applied with shear stress, with low to high foaming and dries clear. As used herein, “unique wasabi-like paste” refers by comparison to, for example a familiar condiment known as wasabi paste made from horseradish, and may include one or more of water or other liquid, mustard, green food dye and other ingredients, wherein the inventive composition is not fibrous or grainy as may be the case with actual wasabi type condiments. The wasabi-like paste may be used as a 3-in-1 composition for acne-prone skin, which includes a daily skin clearing cleanser to be mixed with water, an overnight drying spot treatment in its original form, and/or a mask when layered on damped face.

The compositions may be a cosmetic composition for treating, hydrating, and/or cleansing acne-prone skin.

The present invention also relates to method for treating, hydrating, and/or cleansing acne-prone skin, comprising:
applying onto the skin in need the compositions according to the present invention.

The present invention also relates to a use of a combination of the glycolipid(s), the salicylic acid or its derivative, the cationic polymer, the clay and the solvents comprising water and glycerine for treating, hydrating, and/or cleansing acne-prone skin.

### DETAILED DESCRIPTION OF THE INVENTION

After diligent research, the inventors have surprisingly discovered that compositions comprising at least one glycolipid, at least one salicylic acid or its derivatives having the specific structure of formula (I), at least one cationic polymer, at least one clay, and solvents comprising water and glycerine can treat, hydrate, and/or cleanse acne-prone skin, thus completing the present invention.

In one embodiment, the composition according to the present invention comprises:
(a) at least one glycolipid:
(b) at least one salicylic acid or its derivatives of formula (I):

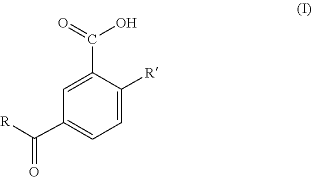

in which:
   the radical R denotes a linear, branched or cyclic, saturated aliphatic chain containing from 2 to 22 carbon atoms; an unsaturated chain containing from 2 to 22 carbon atoms containing one or more double bonds that may be conjugated; an aromatic nucleus linked to the carbonyl radical directly or via saturated or unsaturated aliphatic chains containing from 2 to 7 carbon atoms; said groups possibly being substituted with one or more substituents, which may be identical or different, chosen from halogen atoms, a trifluoromethyl group, hydroxyl groups in free form or esterified with an acid containing from 1 to 6 carbon atoms, or carboxyl groups in free form or esterified with a lower alcohol containing from 1 to 6 carbon atoms;
   R′ is a hydroxyl group or an ester group of formula:

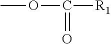

in which R1 denotes a linear or branched, saturated or unsaturated aliphatic chain containing 1 to 18 carbon atoms; and also salts thereof derived from an inorganic or organic base:
(c) at least one cationic polymer selected from a polysaccharide derived from β-linked D-glucosamine or N-acetyl-D-glucosamine;
(d) at least one clay present from about 5% to about 25% by weight, relative to the total weight of the composition;
(e) cosmetically acceptable solvents comprising water, and comprising glycerin present from about 2% to about 40% by weight, relative to the total weight of the composition; and
(f) at least one surfactant present from about 1% to about 20% by weight, relative to the total weight of the composition,
wherein the pH of the composition is from about 3.5 to about 6.5, and
wherein the composition has a wasabi-like paste texture.

In another embodiment, the composition according to the present invention comprises:
at least one rhamnolipid present from 2% by weight to 5% by weight, relative to the total weight of the composition;
(b) at least one salicylic acid or its derivatives or its derivatives of formula (I):

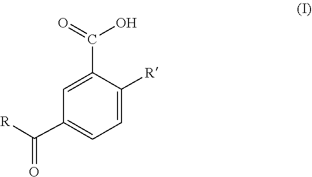

in which:
   the radical R denotes a linear, branched or cyclic, saturated aliphatic chain containing from 2 to 22 carbon atoms; an unsaturated chain containing from 2 to 22 carbon atoms containing one or more double bonds that may be conjugated; an aromatic nucleus linked to the carbonyl radical directly or via saturated or unsaturated aliphatic chains containing from 2 to 7 carbon atoms; said groups possibly being substituted with one or more substituents, which may be identical or different, chosen from halogen atoms, a trifluoromethyl group, hydroxyl groups in free form or esterified with an acid containing from 1 to 6 carbon atoms, or carboxyl groups in free form or esterified with a lower alcohol containing from 1 to 6 carbon atoms;
   R′ is a hydroxyl group or an ester group of formula:

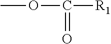

in which R1 denotes a linear or branched, saturated or unsaturated aliphatic chain containing 1 to 18 carbon atoms; and also salts thereof derived from an inorganic or organic base;
the at least one salicylic acid or its derivatives or its derivatives of formula (I) being present from about 1% by weight to about 3% by weight, relative to the total weight of the composition;
(c) at least one cationic polymer comprising chitosan;
(d) at least one clay present from about 5% to about 25% by weight, relative to the total weight of the composition:
(e) cosmetically acceptable solvents comprising water, and comprising glycerin present from about 10% to about 40% by weight, relative to the total weight of the composition; and
(f) at least one surfactant present from about 1% to about 20% by weight, relative to the total weight of the composition,
wherein the pH of the composition is from about 3.5 to about 6.5,
wherein the composition has a wasabi-like paste texture, and
wherein the composition is a 3-in-1 paste formula base for acne-prone skin as a skin cleanser, as a spot treatment, and as a mask.

Hereinafter, the compositions and the method according to the present invention will be explained in a more detailed manner.

### Compositions

The compositions according to the present invention may be a cosmetic composition, alternatively a cosmetic composition for a keratinous substance, alternatively a cosmetic composition for treating, hydrating, and/or cleansing acne-prove skin. The keratinous substance here means a material containing keratin as a main constituent element, and examples thereof include the facial or body skin, scalp, lips, and the like.

The form of the compositions according to the present invention is not limited. In general, the composition according to the present invention can be liquid, semi-liquid, or solid at room temperature (25° C.) and atmospheric pressure (10⁵ Pa). The composition may take various forms, such as, but not limited to, a solution, an aqueous solution, a lotion, a milky lotion, a cream, a gel, a liquid gel, a paste, a serum, a suspension, a dispersion, a fluid, a milk, an emulsion (O/W or W/O form), or the like.

The compositions according to the present invention may be in the form of a paste, such as, but not limited to, a unique wasabi-like paste texture.

The compositions according to the present invention may be used as a cosmetic composition. The composition according to the present invention may be intended for application onto a keratinous substance, such as, but not limited to, skin, such as, but not limited to, facial skin.

In some embodiments, the wasabi-like paste may be used as a 3-in-1 composition for acne-prone skin, which includes a daily skin clearing cleanser to be mixed with water, an overnight drying spot treatment in its original form, and/or a mask when layered on damped face.

The compositions can be used as a daily skin clearing cleanser, to be mixed with water, applied and rubbed on to the skin, and, after a short period of time, rinsed off with water. For spot treatment, the compositions can be dotted onto skin in its original form and rinsed off with water after a prolonged period of time, e.g., overnight. In the form of a mask, the compositions can be layered on damped face and retained on the skin for a selected period of time, then rinsed off water.

In one embodiment, the composition according to the present invention comprises (a) at least one glycolipid: (b) at least one salicylic acid or its derivatives according to formula (I): (c) at least one cationic polymer, (d) at least a clay, and (e) cosmetically acceptable solvents comprising water, and comprising glycerin present from about 2% to about 40%, relative to the total weight of the composition, and wherein the pH of the composition is from about 3.5 to about 6.5.

In an alternative embodiment a plant-based thickener may be added to any compositions described herein, or may be substituted for any clay disclosed herein in equal proportions by weight. In an alternative embodiment, the compositions disclosed herein may be free of plant-based thickeners.

In another embodiment, the composition according to the present invention comprises: (a) at least one rhamnolipid present from 2% by weight to 5% by weight, relative to the total weight of the composition: (b) at least one salicylic acid or its derivatives present from about 1% by weight to about 3% by weight, relative to the total weight of the composition: (c) at least one cationic polymer comprising chitosan: (d) at least one clay comprising kaolin; and (e) cosmetically acceptable solvents comprising water, and comprising glycerin present from about 10% to about 30% by weight, relative to the total weight of the composition: wherein the pH of the composition is from about 3.5 to about 6.5: wherein the composition is suitable for 3-in-1 paste formulas for acne-prone skin as a skin cleanser, as a spot treatment, and as a mask.

The ingredients in the composition will be described in a detailed manner below.

### Glycolipid

The compositions according to the invention comprise one or more glycolipids. The amount of the glycolipid(s), including rhamnolipids, may be 0.1% to 10% by weight, alternatively 1% to 7%, including 1%, 2%, 3%, 4% 5%, 6%, and 7%, by weight, alternatively 2% to 5% by weight, alternatively 3% by weight, relative to the total weight of the composition.

The term “glycolipid” is understood as meaning a compound formed from a lipid to which are attached one or more sugar compounds.

The one or more glycolipids may be selected from rhamnolipids, sophorolipids, glucolipids, trehalolipids, cellobiose lipids and mixtures thereof.

The one or more glycolipids may be selected from rhamnolipids, sophorolipids and mixtures thereof.

The one or more glycolipids may be glucolipids, which contain a glucose moiety and can be represented by the general formula (I):

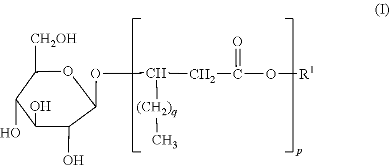

in which:
   R¹ represents a hydrogen atom or a cation;
   p denotes an integer ranging from 1 to 4; and
   q denotes an integer ranging from 4 to 10, alternatively equal to 6.

The glucolipids can be produced by the bacterium Alcaligenes sp. MM1. The appropriate fermentation methods are reviewed by M. Schmidt in his doctoral thesis (1990), Technical University of Braunschweig, and by Schulz et al. (1991) Z. Naturforsch., 46C, 197-203. The glucolipids are recovered from the fermentation broth by solvent extraction using diethyl ether or a dichloromethane:methanol or chloroform:methanol mixture.

### Sophorolipids:

The one or more glycolipids may be sophorolipids, which contain a sophorose moiety and can be represented by the general formula (II):

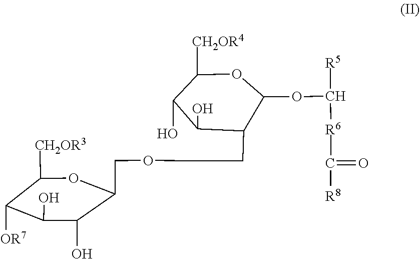

in which:
   R³ and R⁴ individually represent a hydrogen atom or an acetyl group,
   R⁵ represents a saturated or unsaturated, hydroxylated or non-hydroxylated hydrocarbon group having from 1 to 9 carbon atoms, alternatively methyl,
   R⁶ represents a saturated or unsaturated, hydroxylated or non-hydroxylated hydrocarbon group having from 1 to 19 carbon atoms, with the proviso that the total number of carbon atoms in the groups R⁵ and R⁶ does not exceed 20, alternatively from 14 to 18.

Sophorolipids may be incorporated into the composition according to the invention either in the form of the open-chain free acid, where R7 represents a hydrogen atom and R8 represents a hydroxy group OH, or in its lactone form, where a lactone ring is formed between R7 and R8, as indicated by formula (III):

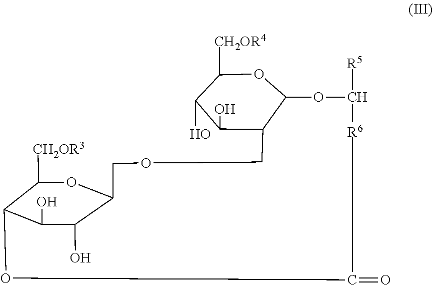

in which:
   R³, R⁴, R5 and R⁶ are as defined above,
      with the proviso that at least one of R³ and R⁴ represents an acetyl group.

The sophorolipids can be produced by yeast cells, such as, but not limited to, Torulopsis apicola and Torulopsis bombicola cells. The fermentation process generally uses sugars and alkanes as substrates.

Appropriate fermentation methods are reviewed in A. P. Tulloch, J. F. T. Spencer and P. A. J. Gorin, Can. J. Chem. (1962), 40, 1326, and U. Gobbert, S. Lang and F. Wagner, Biotechnology Letters (1984), 6 (4), 225. The resulting product is a mixture of various open-chain sophorolipids and of sophorolipid lactones that may be used in the form of mixtures, or the required form may be isolated.

It is possible to use as sophorolipids such as, but not limited to, that sold under the Sopholiance S name by Givaudan and that sold under the BioToLife name by BASF.

### Trehalolipids:

The one or more glycolipids may be trehalolipids, which contain a trehalose fragment and can be represented by the general formula (IV):

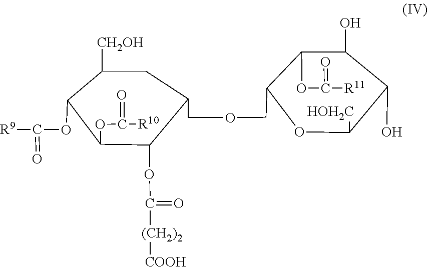

in which:
   R⁹, R¹⁰ and R¹¹ individually represent a saturated or unsaturated, hydroxylated or non-hydroxylated hydrocarbon radical having from 5 to 13 carbon atoms.

The trehalolipids can be produced by bacterial fermentation using the marine bacterium Arthrobacter sp. Ek 1 or the freshwater bacterium Rhodococcus erythropolis. Appropriate fermentation methods are provided by Ishigami et al. (1987), J. Jpn. Oil Chem. Soc., 36, 847-851, Schultz et al. (1991), Z. Naturforsch., 46C, 197-203, and Passeri et al. (1991), Z. Naturforsch., 46C, 204-209.

### Cellobiose Lipids:

The one or more glycolipids may be cellobiose lipids, which contain a cellobiose fragment and can be represented by the general formula (V):

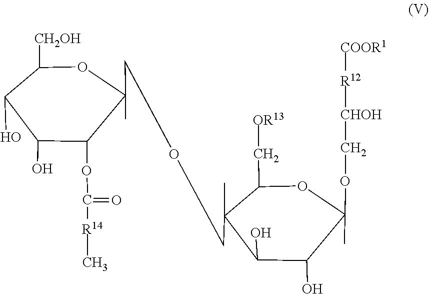

in which:
   R¹ represents a hydrogen atom or a cation,
   R¹² represents a saturated or unsaturated, hydroxylated or non-hydroxylated hydrocarbon radical having from 9 to 15 carbon atoms, alternatively 13 carbon atoms,
   R¹³ represents a hydrogen atom or an acetyl group;
   R¹⁴ represents a saturated or unsaturated, hydroxylated or non-hydroxylated hydrocarbon radical having from 4 to 16 carbon atoms.

The cellobiose lipids can be produced by cells of fungi of the genus Ustilago. Appropriate fermentation processes are provided by Frautz, Lang and Wagner (1986), Biotech. Letts., 8, 757-762.

### Rhamnolipids:

The one or more glycolipids may be rhamnolipids.

The composition according to the invention may include one or more rhamnolipids.

Rhamnolipids are glycolipids produced by various bacterial species. They consist of one rhamnose fragment (mono-rhamnolipid) or of two rhamnose fragments (di-rhamnolipid) linked by a glycosidic bond to one, two or three chains of β-hydroxylated fatty acids linked to one another by an ester bond.

More specifically, these mono-rhamnolipids and di-rhamnolipids correspond to the following formula (VI):

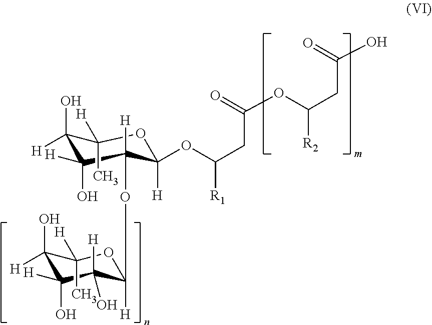

in which:
   m denotes an integer equal to 2, 1 or 0;
   n denotes an integer equal to 1 or 0; and
   R₁ and R₂, each independently represent identical or different hydrocarbon radicals having from 2 to 24 carbon atoms, alternatively from 5 to 13 carbon atoms, that are branched or unbranched, substituted or unsubstituted, such as, but not limited to, hydroxy-substituted, and saturated or unsaturated, alternatively a singly, doubly or triply unsaturated alkyl radical.

Thus, when n is equal to 0, the formula (VI) protects mono-rhamnolipids and, when n is equal to 1, it protects di-rhamnolipids.

The composition according to the invention may include at least one di-rhamnolipid.

The composition according to the invention may include at least one di-rhamnolipid of formula (VI) in which:
m denotes an integer equal to 2, 1 or 0;
n denotes an integer equal to 1; and
R₁ and R₂, each independently represent identical or different hydrocarbon radicals having from 2 to 24 carbon atoms, alternatively from 5 to 13 carbon atoms, that are branched or unbranched, substituted or unsubstituted, such as, but not limited to, hydroxy-substituted, and saturated or unsaturated, alternatively a singly, doubly or triply unsaturated alkyl radical, and also the salts thereof, solvates thereof and optical isomers thereof.

The glycosidic bond between the two rhamnose fragments may be in the alpha or beta configuration, alternatively in the alpha configuration.

In the context of the invention,
the salts of the di-rhamnolipids of formula (VI) may be the carboxylate salts thereof with an organic or inorganic cation and especially with a cation selected from sodium, potassium, calcium and ammonium;
the solvated forms of the di-rhamnolipids of formula (VI) may be those solvated with one or more molecules of water or of organic solvents, such as, but not limited to, a hydrate or a solvate of a linear or branched alcohol, such as, but not limited to, ethanol or isopropanol, the optically active carbon atoms of the fatty acids in one embodiment being in the form of the R enantiomers; and
the term “alkyl” radical denotes a saturated, linear or branched aliphatic group: such as, but not limited to, a C₁-C₂₀ alkyl group having a linear or branched hydrocarbon chain of 1 to 20 carbon atoms, such as, but not limited to, a methyl, ethyl, propyl, isopropyl, butyl, isobutyl, tert-butyl, pentyl, hexyl, heptyl, octyl, nonyl, decyl, undecyl, dodecyl, tridecyl, tetradecyl, pentadecyl, hexadecyl, heptadecyl, octadecyl, nonadecyl or eicosyl.

The composition according to the invention may include at least one di-rhamnolipid of formula (VI) in which:
m denotes an integer equal to 2, 1 or 0:
n denotes an integer equal to 1; and
R₁ and R₂, which are identical or different, are selected from pentenyl, hexenyl, heptenyl, octenyl, nonenyl, decenyl, undecenyl, dodecenyl and tridecenyl radicals and radicals of formula —(CH₂)₆CH₃, with o denoting an integer ranging from 1 to 23, alternatively from 3 to 15, alternatively from 4 to 12.

According to one embodiment of the invention, the composition according to the invention comprises at least one di-rhamnolipids of general formula (VI) in which m is equal to 1.

According to one embodiment of the invention, the composition according to the invention comprises a mixture of at least two, alternatively at least three, di-rhamnolipids of general formula (VI) in which m may be equal to 1.

According to another embodiment of the invention, the composition according to the invention comprises a mixture comprising at least one mono-rhamnolipid.

In a further embodiment, the composition according to the invention comprises at least one dirhamnolipid of the following formula (VII):

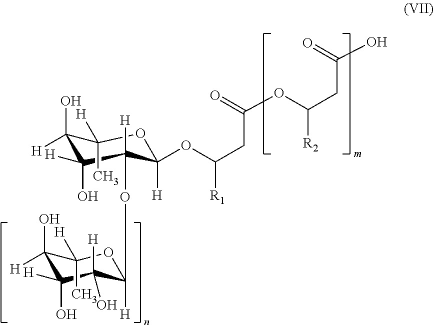

in which:
   m denotes an integer equal to 2, 1 or 0; alternatively, m is equal to 1,
   n denotes an integer equal to 1,
   R₁ is a —(CH₂)ₚ—CH₃ radical, with p being an integer varying from 1 to 23, alternatively from 4 to 12,
   R₂ is a —(CH₂)_{q}—CH₃ radical, with q being an integer varying from 1 to 23, alternatively from 4 to 12,
and also the salts thereof, solvates thereof and optical isomers thereof.

By way of illustration and without limiting the di-rhamnolipids of formula (VII) that may be suitable for the invention, mention may be made of the compounds of formula di-RL-CXCY, such as are defined in Table 1 below.

The formula di-RL-CXCY is an alternative way of writing in order to represent a di-rhamnolipid (di-RL) functionalized by two radicals R₁ and R₂ respectively represented by the symbols CX and CY, the integers X and Y being respectively equal to p+4 and q+4.

TABLE 1

| Composés | Di-RL-CXCY | p | q |
|---|---|---|---|
| 1 | diRL-C8C8 | 4 | 4 |
| 2 | diRL-C8C10 | 4 | 6 |
| 3 | diRL-C10C8 | 6 | 4 |
| 4 | diRL-C10C10 | 6 | 6 |
| 5 | diRL-C10C12 | 6 | 8 |
| 6 | diRL-C12C10 | 8 | 6 |
| 7 | diRL-C12C12 | 8 | 8 |
| 8 | diRL-C12C14 | 8 | 10 |
| 9 | diRL-C14C12 | 10 | 8 |
| 10 | diRL-C14C14 | 10 | 10 |
| 11 | diRL-C14C16 | 10 | 12 |
| 12 | diRL-C16C14 | 12 | 10 |
| 13 | diRL-C16C16 | 12 | 12 |

According to one embodiment, the composition according to the invention comprises at least one di-rhamnolipid of formula (VII) in which p and q are identical and equal to 6 and m is equal to 1, also referred to as di-RL-C10C10, or one of the salts, solvates and optical isomers thereof.

In one embodiment, the di-rhamnolipid of formula (VII) in which p and q are identical and equal to 6 and m is equal to 1 is present in the composition according to the invention in a proportion of at least 50% by weight, alternatively from 51% to 85% by weight, relative to the total weight of rhamnolipids.

According to another embodiment, the composition according to the invention comprises at least one di-rhamnolipid of formula (VII) in which m is equal to 1, p is equal to 6 and q is equal to 8.

According to another embodiment, the composition according to the invention comprises at least one di-rhamnolipid of formula (VI) in which n and m are equal to 1, R₁ represents a —(CH₂)ₒCH₃ radical, with o being an integer varying from 4 to 12, and R₂ is selected from the pentenyl, hexenyl, heptenyl, octenyl, nonenyl, decenyl, undecenyl, dodecenyl and tridecenyl radicals; alternatively, R₁ represents a —(CH₂)₆CH₃ radical and R₂ a nonenyl radical.

According to another embodiment, the composition according to the invention comprises a mixture of at least two, alternatively at least three, di-rhamnolipids of formula (VI) or of formula (VII) selected from:
a di-rhamnolipid of formula (VII) in which p and q are identical and equal to 6 and m is equal to 1;
a di-rhamnolipid of formula (VII) in which m is equal to 1, p is equal to 6 and q is equal to 8; and
at least one di-rhamnolipid of formula (VI) in which n and m are equal to 1, R₁ represents a —(CH₂)ₒCH₃ radical, with o being an integer varying from 4 to 12, and R₂ is selected from the pentenyl, hexenyl, heptenyl, octenyl, nonenyl, decenyl, undecenyl, dodecenyl and tridecenyl radicals; alternatively, R₁ represents a —(CH₂)₆CH₃ radical and R2 a nonenyl radical.

In one embodiment, the composition according to the invention comprises a mixture of at least two, alternatively at least three, di-rhamnolipids of formula (VI) or of formula (VII) selected from:
at least 50% by weight, alternatively from 51% to 85% by weight of a di-rhamnolipid of formula (VII) in which p and q are identical and equal to 6 and m is equal to 1, relative to the total weight of rhamnolipids:
from 0.5% to 25% by weight, alternatively from 5% to 15% by weight, of a dirhamnolipid of formula (VII) in which p is equal to 6, q is equal to 8 and m is equal to 1, relative to the total weight of rhamnolipids; and
from 0.5% to 15% by weight, alternatively from 3% to 12% by weight, alternatively from 5% to 10% by weight, of a dirhamnolipid of formula (VI) in which n and m are equal to 1, R₁ represents a —(CH₂)₆CH₃ radical and R2 represents a nonenyl radical, relative to the total weight of rhamnolipids.

As specified above, rhamnolipids are customarily prepared by processes known to those skilled in the art starting from bacterial producers, such as, but not limited to, Pseudomonas.

Appropriate fermentation methods are reviewed by D. Haferburg, R. Hommel, R. Claus and H. P. Kleber in Adv. Biochem. Ing./Biotechnol. (1986), 33, 53-90, and by F. Wagner, H. Bock and A. Kretschmar in Fermentation (ed. R. M. Lafferty) (1981), 181-192, Springer Verlag, Vienna.

Use may be made, as rhamnolipid, of the one sold under the name Rheance One by Evonik (INCI name: glycolipids).

The amount of rhamnolipids, may be 0.1% to 10% by weight, alternatively 1% to 7%, including 1%, 2%, 3%, 4% 5%, 6%, and 7%, by weight, alternatively 2% to 5% by weight, alternatively 3% by weight, relative to the total weight of the composition.

### Salicylic Acid or its Derivatives

The composition according to the present invention comprises at least one salicylic acid or its derivatives according to formula (I). A single type of the salicylic acid or its derivative may be used, but two or more different types of the salicylic acid or its derivatives may be used in combination.

The salicylic derivatives in accordance with the present invention correspond to formula (I):

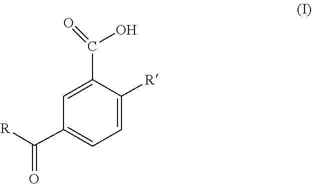

in which:
   the radical R denotes a linear, branched or cyclic, saturated aliphatic chain containing from 2 to 22 carbon atoms; an unsaturated chain containing from 2 to 22 carbon atoms containing one or more double bonds that may be conjugated; an aromatic nucleus linked to the carbonyl radical directly or via saturated or unsaturated aliphatic chains containing from 2 to 7 carbon atoms; said groups possibly being substituted with one or more substituents, which may be identical or different, chosen from halogen atoms, a trifluoromethyl group, hydroxyl groups in free form or esterified with an acid containing from 1 to 6 carbon atoms, or carboxyl groups in free form or esterified with a lower alcohol containing from 1 to 6 carbon atoms;
   R′ is a hydroxyl group or an ester group of formula:

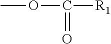

in which R1 denotes a linear or branched, saturated or unsaturated aliphatic chain containing 1 to 18 carbon atoms;
and also salts thereof derived from an inorganic or organic base.

In one embodiment, the R radical in formula (I) denotes a linear, branched or cyclic, saturated aliphatic chain containing from 3 to 11 carbon atoms: an unsaturated chain containing from 3 to 17 carbon atoms and comprising one or more conjugated or unconjugated double bonds: it being possible for said hydrocarbon-based chains to be substituted with one or more substituents, which may be identical or different, chosen from halogen atoms, a trifluoromethyl group, hydroxyl groups in free form or esterified with an acid containing from 1 to 6 carbon atoms, or a carboxyl function in free form or esterified with a lower alcohol containing from 1 to 6 carbon atoms.

In one embodiment, the R radical in formula (I) denotes a linear or branched alkyl or alkenyl group, alternatively alkyl group, containing 2 or more, alternatively 3 or more, alternatively 4 or more, alternatively 5 or more, alternatively 6 or more carbon atoms, and/or 22 or less, alternatively 18 or less, alternatively 14 or less, alternatively 12 or less, alternatively 10 or less carbon atoms.

In one embodiment, R′ in formula (I) is a hydroxyl group or an ester group of formula:

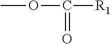

in which R1 denotes a radical —(CH₂)ₙ—CH₃ where n is a number ranging from 0 to 14.

The salicylic acid derivatives may include those according to formula (I) in which the R radical is a C₃-C₁₀ alkyl group and/or R′ denotes hydroxyl.

Other advantageous compounds are those in which R represents a chain derived from caprylic, linoleic, linolenic or oleic acid.

Another group of suitable salicylic acid derivatives is constituted of compounds in which the R radical denotes a C3-C10 alkyl group bearing a carboxyl function in free form or esterified with a lower alcohol containing from 1 to 6 carbon atoms and R′ denotes hydroxyl.

The salicylic acid or its derivatives of formula (I) that may be used according to the present invention are in particular described in patents U.S. Pat. Nos. 6,159,479 and 5,558,871, FR 2,581,542, U.S. Pat. No. 4,767,750, EP 378 936, U.S. Pat. Nos. 5,267,407, 5,667,789, 5,580,549 and EP-A-570,230.

Suitable salicylic acid derivatives of formula (I) include 5-n-octanoylsalicylic acid (or capryloyl salicylic acid): 5-n-decanoylsalicylic acid: 5-n-dodecanoylsalicylic acid: 5-n-heptyloxysalicylic acid, and the corresponding salts thereof. The derivative in question may be 5-n-octanoylsalicylic acid.

For the purposes of the present invention, the salts of the salicylic acid or its derivatives are also considered. As the salts derived from inorganic bases, mention may be made of those derived from alkali metal or alkaline-earth metal hydroxylated bases, for instance sodium hydroxide or potassium hydroxide, and ammonia. As regards the salts derived from the organic bases, mention may be made of those derived from bases of amine or alkanolamine type.

The amount of the salicylic acid or its derivatives may be from 0.1% to 5% by weight, alternatively from 1% to 3% by weight, alternatively 2% by weight, relative to the total weight of the composition.

The compositions disclosed herein may have any suitable weight ratio of glycolipid to salicylic acid or its derivatives, including, but not limited to, 3:1 to 1:3, alternatively 2:1 to 1:2, alternatively 1:1 to 2:1, alternatively 1.2:1 to 1.8:1, alternatively 1.4:1 to 1.6:1, alternatively 1.5:1.

### Cationic Polymer

In various embodiments, the acne care composition may comprise at least one cationic polymer selected from nature-based polymers that are polysaccharides, and other natural (i.e., plant, animal, or bacterial based), synthetic, or modified cationic nature-based or synthetic polymers.

The at least one cationic polymer may be selected from the group consisting of chitosan, chitosan oligosaccharide, chitin, cyclodextrin, cationic gelatin, cationic dextran, cationic cellulose, polylysine, polyornithine, histone, collagen, chitosan-cysteine, chitosan-thiobutylamidine, chitosan-thioglycolic acid, and combinations thereof.

Examples of cationic polymers include polysaccharide-based delivery molecules (e.g., chitosan, cyclodextrin, cationic gelatin, cationic dextran, cationic cellulose), cationic peptides and their derivatives (e.g., polylysine, polyornithine), peptide/protein polymers ((e.g., histone, collagen), linear or branched synthetic polymers (e.g., poly brene, polyethyleneimine), natural polymers (e.g., histone, collagen), synthetic dendrimers, cationic thiolated biopolymers (nature-based thiomers or nature-based dendrimers, e.g., chitosan-cysteine, chitosan-thiobutylamidine as well as chitosan-thioglycolic acid).

In some embodiments, the composition may include or exclude any other cationic polymer, including a nature-based polymer or synthetic polymer.

In certain embodiments, the cationic polymer may be a polysaccharide derived from β-linked D-glucosamine and N-acetyl-D-glucosamine. In a further embodiment, the cationic polymer is chitosan.

In various embodiments, chitosan has a molecular weight (MW) in a range from about 1 kDa to about 1000 kDa. In some embodiments, chitosan has a MW that is “low” and is in the range from about 1 kDa to about 20 kDa, or from about 10 kDa to about 20 kDa, or from about 12 kDa to about 18 kDa. In some embodiments, the chitosan has a Chitosan MW=˜27 kDa.

The amount of the chitosan may be from 0.01% to 3% by weight, alternatively from 0.05% to 1% by weight, alternatively 0.1% to 0.2% by weight, including, but not limited to, about 0.1%, 0.15%, and 0.2%, relative to the total weight of the composition.

According to some embodiments, the compositions comprise chitosan present at about 0.1%, by weight, relative to the total weight of the composition.

### Clay

The at least one clay may be selected from the group of clays consisting of kaolinites, smectite, sodium smectite, calcium smectite, illite, chlorite, vermiculite, attapulgite, and combinations thereof.

In some embodiments, the at least one clay may be a smectite clay in the mica family of phylosilicates. In some embodiments, the at least one clay may comprise hectorite. In most embodiments, the at least one clay comprises kaolin.

The amount of the clay may be from 5% to 25% by weight, alternatively from 10% to 20% % by weight, including 10%, 11%, 12%, 13%, 14%, 15%, 16%, 17%, 18% 19% and 20%; alternatively 15% by weight, relative to the total weight of the composition.

### Cosmetically Acceptable Solvents

Compositions according to the disclosure contain at least one cosmetically acceptable solvent. In various embodiments, the cosmetically acceptable solvent may be chosen from water.

### Water:

In accordance with the various embodiments, water is present in the composition in a range from about 10% to about 60%, or from about 20% to about 55%, or from about 25% to about 50%, or from about 28% to about 49%, or any suitable combination, sub-combination, range, or sub-range thereof by weight, based on the weight of the composition. One of ordinary skill in the art, however, will appreciate that other ranges are within the scope of the invention.

Thus, water may be present by weight, based on the weight of the composition, from about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, to about 60 weight percent, including increments and ranges therein and there between.

The water used may be sterile demineralized water and/or a floral water such as, but not limited to, rose water, comflower water, chamomile water or lime water, and/or a natural thermal or mineral water such as, but not limited to, water from Vittel, water from the Vichy basin, water from Uriage, water from La Roche Posay, water from La Bourboule, water from Enghien-les-Bains, water from Saint Gervais-les-Bains, water from Neris-les-Bains, water from Allevar-les-Bains, water from Digne, water from Maizieres, water from Neyrac-les-Bains, water from Lons-le-saunier, water from Eaux Bonnes, water from Rochefort, water from Saint Christau, water from Les Fumades, water from Tercis-les-Bains or water from Avene.

The water phase may also comprise reconstituted thermal water, that is to say a water comprising trace elements such as, but not limited to, zinc, copper, magnesium, etc., reconstituting the characteristics of a thermal water.

### Water-Soluble Solvents:

In accordance with some embodiments, the compositions include at least one water-soluble solvent. The term “water-soluble solvent” is interchangeable with the term “water-miscible solvent” and means a compound that is liquid at 25° C. and at atmospheric pressure (760 mmHg), and it has a solubility of at least 50% in water under these conditions. In some cases, the water-soluble solvent has a solubility of at least 60%, 70%, 80%, or 90% in water under these conditions.

### Glycerin:

In some embodiments, the water-soluble solvent includes glycerin.

The amount of the glycerin may be from 1% to 45% by weight, alternatively from 2% to 40% % by weight, alternatively 10% to 30% by weight, including, but not limited to, 10%, 15%, 20%, 25%, and 30%, relative to the total weight of the composition.

As examples of organic solvents, non-limiting mentions can be made of ethyl alcohol, isopropyl alcohol, propyl alcohol, benzyl alcohol, and phenylethyl alcohol, or glycols or glycol ethers such as, but not limited to, monomethyl, monoethyl and monobutyl ethers of ethylene glycol, propylene glycol or ethers thereof such as, but not limited to, monomethyl ether of propylene glycol, butylene glycol, hexylene glycol, dipropylene glycol as well as alkyl ethers of diethylene glycol, such as, but not limited to, monoethyl ether or monobutyl ether of diethylene glycol.

Other suitable examples of organic solvents are ethylene glycol, propylene glycol, butylene glycol, hexylene glycol, propane diol, and glycerin. The organic solvents can be volatile or non-volatile compounds.

Further non-limiting examples of water-soluble solvents include alkanols (polyhydric alcohols, glycols and polyols) such as, but not limited to, glycerin, 1,2,6-hexanetriol, trimethylolpropane, ethylene glycol, propylene glycol, diethylene glycol, butylene glycol, hexylene glycol, triethylene glycol, tetraethylene glycol, pentaethylene glycol, dipropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, 2-butene-1,4-diol, 2-ethyl-1,3-hexanediol, 2-methyl-2,4-pentanediol, 1,2-hexanediol, 1,2-pentanediol, and 4-methyl-1,2-pentanediol.

Further non-limiting examples of water-soluble solvents include alkyl alcohols having 1 to 4 carbon atoms such as, but not limited to, ethanol, methanol, butanol, propanol, and isopropanol: glycol ethers such as, but not limited to, ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxy butanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether: 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, or mixtures thereof.

In accordance with the various embodiments the amount of the at least one water-soluble solvent is from about 0.1% to about 25%, or from about 0).1% to about 2%, or from about 0.1% to about 1%, or from about 0.1% to about 0.8%, or from about 0.1% to about 0.5%, or from about 1% to about 20%, or from about 1% to about 10%, or from about 2% to about 8%, or any suitable combination, sub-combination, range, or sub-range thereof by weight, based on the weight of the composition. One of ordinary skill in the art, however, will appreciate that other ranges are within the scope of the invention.

In some embodiments, the compositions include more than one water soluble solvent, each water soluble solvent presents in an amount as set forth herein above, wherein each different water soluble solvent may be present within one of the ranges selected from the ranges set forth herein above.

Thus, water-soluble solvents may be present by weight, based on the total weight of the composition, from about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24 to about 25 weight percent, including increments and ranges therein and there between.

### Surfactants

In various embodiments, the compositions further comprise the surfactant sodium cocoyl isethionate.

In some embodiments, the compositions may include, or alternatively may be substantially free from, or exclude any one or more of nonionic, cationic, or anionic surfactants. In some embodiments, the compositions exclude all surfactants except sodium cocoyl isethionate.

In some embodiments, the compositions also include at least one additional surfactant selected from glyceryl oleate, glyceryl stearate, and cetyl alcohol. In some embodiments, the composition includes at least glyceryl stearate SE in addition to sodium cocoyl isethionate.

The concentration of sodium cocoyl isethionate present in the compositions is from about 1% to about 20%, or from about 2% to about 18%, or from about 5% to about 15%, or from about 8% to about 12%, or about 10%, or any suitable combination, sub-combination, range, or sub-range thereof by weight, based on the weight of the composition. One of ordinary skill in the art, however, will appreciate that other ranges are within the scope of the invention.

Thus, sodium cocoyl isethionate is present, by weight, based on the total weight of the composition, from about 1.0, 1.1, 1.2, 1.3, 1.4, 1.5, 1.6, 1.7, 1.8, 1.9, 2.0, 3, 4, 5, 6, 7, 8, 9, 10, 11, 12, 13, 14, 15, 16, 17,18, 19 to about 20 weight percent, including increments and ranges therein and there between.

When present, an additional surfactant, such as, but not limited to, glyceryl stearate SE, is present in the compositions is from about 1% to about 20%, or from about 2% to about 18%, or from about 5% to about 15%, or from about 8% to about 12%, or from about 2% to about 6%, or about 5%, or any suitable combination, sub-combination, range, or sub-range thereof by weight, based on the weight of the composition. One of ordinary skill in the art, however, will appreciate that other ranges are within the scope of the invention.

Thus, an additional surfactant, when present, is present, by weight, based on the total weight of the composition, from about 1.0, 1.1, 1.2, 1.3, 1.4, 1.5, 1.6, 1.7, 1.8, 1.9, 2.0, 3, 4, 5, 6, 7, 8, 9, 10, 11, 12, 13, 14, 15, 16, 17,18, 19 to about 20 weight percent, including increments and ranges therein and there between.

### Additives

In some embodiments, there may be one or more optional actives or other ingredients (herein, “additives”) present in the compositions, including, but not limited to, anionic, cationic, nonionic or amphoteric polymers, anionic, cationic, nonionic or amphoteric surfactants, oils, hydrophobic organic solvents, gums, resins, thickeners, dispersants, antioxidants, buffers, organic or inorganic fillers, preserving agents, such as, but not limited to, phenoxyethanol, fragrances, neutralizers, antiseptics, UV-screening agents, cosmetic active agents such as, but not limited to, vitamins, moisturizers, emollients or collagen-protecting agents, anti-microbials, chelating agents, penetrants, sequestrants, fragrances, skin care actives such as, but not limited to, ceramides, sodium lauroyl lactylate, ceramide np, ceramide ap, phytosphingosine, cholesterol, xanthan gum, carbomer, ceramide eop, opacifiers, alpha-hydroxy acids, organic and inorganic UV filters, citric acid, phenylethyl resorcinol, hydroxypropyl tetrahydropyrantriol, hydroxyacetophenone, phenolic compounds, chalcones, flavones, flavanones, flavanols, flavonols, dihydroflavonols, isoflavonoids, neoflavonoids, catechins, anthocyanidins, tannins, lignans, aurones, stilbenoids, curcuminoids, alkylphenols, betacyanins, capsacinoids, hydroxy benzoketones, methoxyphenols, naphthoquinones, phenolic terpenes, resveratrol, curcumin, pinoresinol, ferulic acid, hydroxytyrosol, cinnamic acid, caffeic acid, p-coumaric acid, baicalin (Scutellaria Baicalensis root extract), pine bark extract (Pinus Pinaster bark/bud extract), ellagic acid, hyaluronic acid and its derivatives, escin (also known as Aescin, a mixture of saponins with anti-inflammatory, vasoconstrictor and vasoprotective effects found in Aesculus hippocastanum), retinol, niacinamide, vitamins and vitamin derivatives, tocopherol, ascorbic acid, or combinations thereof.

Suitable antiseptics may include, but are not limited to, ethanol, triclosan, piroctone olamine, triclocarban, paraben, acrinol, alkyldiaminoglycine or a salt thereof, povidone iodine, potassium iodide, iodine, 1,2-pentanediol, halocarban, 3,4,4-trichlorocarbanilide, triethyl citrate, resorcin, hexachlorophene, silver-supported zeolite, silver-supported silica, gluconic acid, benzoic acid, sodium benzoate, undecylene acid, salicylic acid, sorbic acid or a salt thereof, dehydroacetic acid or a salt thereof, methyl paraoxy benzoate, ethyl paraoxy benzoate, propyl paraoxy benzoate, isopropyl paraoxybenzoate, butyl paraoxybenzoate, isobutyl paraoxybenzoate, benzyl paraoxybenzoate: phenols such as, but not limited to, isopropylmethylphenol, chlorhexidine gluconate, cresol, timole, parachlorophenol, phenylethyl alcohol, phenylphenol, sodium phenylphenol, sodium phenoxyethanol, phenoxydiglycol, phenol, benzyl alcohol: tertiary ammonium salt compounds such as, but not limited to, stearyltrimethylammonium chloride, cetyltrimethylammonium chloride, cetylpyridinium chloride, benzalconium chloride, benzethonium chloride, methylbenzethonium chloride, lauryltrimethylammonium chloride, alkylisoquinolium bromide, domiphenone bromide: plant extracts such as, but not limited to, tea extract, hinokithiol, apple extract: chloramine T, chlorhexidine, benzoic acidion, or mixtures thereof. Antiseptics may be used in any suitable amount, including, but not limited to, an amount ranging from 0.001% to 10% by weight, alternatively from 0.01% to 5% by weight, alternatively from 0.1% to 3% by weight, relative to the total weight of the composition.

Although the aforementioned optional additives are given as an example, it will be appreciated that other optional components compatible with cosmetic applications known in the art may be used. Further, any one of the aforementioned additives may be excluded.

In some embodiments, the compositions include at least one additive selected from the group consisting of sodium hydroxide, niacinamide, lactic acid, cetyl alcohol, trisodium ethylenediamine disuccinate, ceramides, and a combination thereof.

In some embodiments, the composition includes additives comprising sodium hydroxide (0.3-0.5%), niacinamide (2%), lactic acid (0.05%), cetyl alcohol (2.5%), trisodium ethylenediamine disuccinate (0.5-1%), and ceramides (sodium lauroyl lactylate (and) ceramide NP (and) ceramide AP (and) phytosphingosine (and) cholesterol (and) xanthan gum (and) carbomer (and) ceramide EOP) (*final active concentration 0.25%)).

### Niacinamide

The compositions according to the present invention may comprise niacinamide present at about from about 0.1 to about 10% by weight, alternatively from about 0.5% to 5% by weight, alternatively from about 1% to about 3% by weight, including, but not limited to, 1%, 2%, and 3% by weight, alternatively about 2% by weight, relative to the total weight of the composition.

In accordance with the various embodiments, the amounts of additives, such as, but not limited to, actives and other components, that may be present in the compositions can range from about 0.001% to about 50%, or from about 0.5% to about 30%, or from about 1.5% to about 20%, and from about 5% to about 15%, or any suitable combination, sub-combination, range, or sub-range thereof by weight, based on the weight of the acne care composition.

Thus, one or a combination of additives may be present in the compositions, by weight, based on the weight of the composition, each one or the combination present from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0 0.80, 0.90, 1.0, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 to about 50 weight percent, including increments and ranges therein and there between.

### Thickener

In various embodiments, the compositions may comprise at least one stabilizer and/or thickening agent (thickener). The thickener may be thickening polymers. Non-limiting examples of thickening polymers include sclerotium gum, xanthan gum, carrageenan, acacia, agar, algin, alginic acid, ammonium alginate, amylopectin, calcium alginate, sodium alginate, calcium carrageenan, carnitine, carrageenan, dextrin, gelatin, gellan gum, guar gum, tragacanth gum, acacia gum, Arabic gum, guar hydroxy propyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propy lene glycol alginate, sodium carboxy methyl dextran, sodium carrageenan, tragacanth gum, xanthan gum, modified xanthan gum, biosacharide gum, chitin, levan, elsinan, collagen, gelatin, zein, gluten, soy protein, casein, and mixtures thereof.

According to some embodiments, the composition comprises one or more of carrageenan, xanthan gum, and sclerotium gum, or a combination thereof, present in a range from about 0.1% to about 5%, or at about 1%, by weight of the composition.

### Carrageenan:

According to some embodiments, the acne care composition comprises carrageenan as a stabilizer/thickening agent, present at about 1%, by weight of the composition.

The at least one thickener, when present, is present in the composition from about 0.1% to about 5%, or from about 0.2% to about 3%, or from about 0.5% to about 2%, or about 1%, by weight of the composition, or any suitable combination, sub-combination, range, or sub-range thereof by weight, based on the weight of the acne care composition. One of ordinary skill in the art, however, will appreciate that other ranges are within the scope of the invention.

Thus, the at least one thickener may be present, by weight, based on the total weight of the composition, from about 0.05, 0.06, 0.07, 0.08, 0.90, 0.1, 0.2, 0.3, 0.4, 0.5, 0.6, 0.7, 0.8, 0.9, 1.0, 2, 3, 4 to about 5 weight percent, including increments and ranges therein and there between.

### pH Adjusting Agent

The pH of the composition according to the present invention may be adjusted to the desired value using at least one pH adjusting agent, such as, but not limited to, an acidifying or a basifying agent, which are commonly used in cosmetic products.

The pH of the composition is from about 3.5 to about 6.5, alternatively from about 4 to 6, alternatively from about 4.5 to 5.5, alternatively pH is about 5.5 or about 5.0.

Among the acidifying agents, mention may be made, by way of example, of mineral or organic acids such as, but not limited to, hydrochloric acid, ortho-phosphoric acid, sulfuric acid, carboxylic acids such as, but not limited to, acetic acid, tartaric acid, citric acid, and lactic acid, and sulfonic acids.

Among the basifying agents, mention may be made, by way of example, of hydroxides of an alkali metal or an alkaline-earth metal, for instance sodium hydroxide or potassium hydroxide: quaternary ammonium hydroxides and guanidinium hydroxide: alkali metal silicates, such as, but not limited to, sodium metasilicates; amino acids, such as, but not limited to, basic amino acids, such as, but not limited to, arginine, lysine, omithine, citrulline and histidine: carbonates and bicarbonates, such as, but not limited to, of a primary amine, secondary amine or tertiary amine, of an alkali metal or alkaline-earth metal, or of ammonium; and the compounds of the following formula:

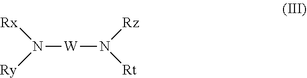

in which:
   W is a C₁-C₆ alkylene residue optionally substituted with a hydroxyl group or a C₁-C₆ alkyl group; and
   Rx, Ry, Rz and Rt, which may be identical or different, represent a hydrogen atom or a C₁-C₆ alkyl, C₁-C₆ hydroxyalkyl or C₁-C₆ aminoalkyl group. Mention may especially be made of 1,3-diaminopropane, 1,3-diamino-2-propanol, spermine and spermidine.

The pH adjusting agent(s) may be used in an amount ranging from 0.001% to 10% by weight, alternatively from 0.01% to 5% by weight, alternatively from 0.1% to 3% by weight, relative to the total weight of the composition.

The composition according to the present invention can be prepared by mixing the above-described essential and optional ingredients in a conventional manner. In the case that at least one of the above ingredients is solid at room temperature, the ingredient can be heated until it is dissolved. It is possible to further comprise mixing any of the optional ingredients and heating the composition until an ingredient is dissolved.

Method for Treating, Hydrating and/or Cleansing Acne-Prone Skin

The present invention also relates to a method for treating, hydrating, and/or cleansing acne-prone skin including scalp, lips, in such as, but not limited to, facial skin, comprising applying onto the acne-prone skin a composition comprising:
(a) at least one glycolipid:
(b) at least one salicylic acid or its derivatives of formula (I) above:
(c) at least one cationic polymer selected from a polysaccharide derived from β-linked D-glucosamine or N-acetyl-D-glucosamine:
(d) at least one clay:
(e) cosmetically acceptable solvents comprising water, and comprising glycerin present from about 2% to about 40% by weight, relative to the total weight of the composition; and
   wherein the pH of the composition is from about 3.5 to about 6.5.

Treating acne-prone skin in present invention means reduction of sebum, or deposition of salicylic acid or its derivatives, or reduction of viable cells of Cutibacterium acnes.

Cleanability of acne-prone skin is calculated as below:

$$\begin{matrix}Cleansability = \frac{{De}_{sample cleaned} - {De}_{black reference}}{{De}_{clean reference} - {De}_{black reference}} \\ {D}_{e} = \sqrt{{D}_{l}^{2} + {D}_{a}^{2} + {D}_{b}^{2}}\end{matrix}$$

*De: color difference in a rectangular coordinate system
*Black reference: bioskin with sebollution applied following pore deep clean protocol
*Clean reference: clean bioskin w/o any sebollution

The composition is generally applied on acne-prone skin with hands, fingers, or an applicator. The present invention may comprise an optional step of rinsing the composition from the keratinous substance after it is applied.

In one embodiment, the composition is a base for a 3-in-1 paste formula for acne-prone skin as a skin cleanser, as a spot treatment, and as a mask. As a base for 3-in-1 paste formula, a single composition forms a daily skin clearing cleanser when mixed with water, is suitable as an overnight drying spot treatment when applied without adulteration, and forms a deeply purifying mask when layered on a damped face. A base for a 3-in-1 paste formula is optimized for physical attributes including spreadability, drying rate, miscibility with water, foaming, rinsibility, appearance, or combinations thereof such that these attributes are at least acceptable, alternatively fair, alternatively good, for the three uses. In a further embodiment, the 3-in-1 paste formula includes optimal concentrations of a clay, such as, but not limited to, kaolin, a solid mild surfactant, such as, but not limited to, sodium cocoyl isethionate, and a water-soluble solvent, such as, but not limited to, glycerin, the combination of which may provide fast-drying, sebum control, and hydration.

The base for a 3-in-1 paste formula when mixed with water to form the daily skin clearing cleanser may provide foaming (lathering), a revitalizing scent, easy movement on skin, pore cleansing, cleansing sensation, dirt removal, oil removal, ease of use, ease of removal, brightening, or combinations thereof.

The base of a 3-in-1 paste formula, used without adulteration as a spot treatment may provide a sufficiently drying characteristic so as to remain in place on skin, a lack of residue, a lack of rubbing off, reduction or prevention of blemishes or acne, a drying time of ten minutes or less at room temperature, ease of removal, or combinations thereof.

The base of a 3-in-1 paste formula, used as a mask layered on damp skin may dry to a provide a dry sensation on skin, become stiff and tight on skin, reduce or eliminate acne, ease of application, ease of removal, a tightening sensation to skin, a thinner layer than known clay masks in the art, or combinations thereof.

The composition used in the method according to the present invention may include any of the optional ingredients as explained above for the composition according to the present invention.

### EXAMPLES

The present invention will be described in more detail by way of examples which however should not be construed as limiting the scope of the present invention.

### Examples 1 to 11 of Present Invention

Compositions of Examples 1 to 11 were prepared by mixing the raw materials as listed in Table 2 with a magnetic stirrer. The numerical values for the amounts of the raw materials shown in Table 2 are all based on “% by weight.” Note that rhamnolipids were obtained from EVONIK (trade name: RHEANCE One, INCI name: glycolipids), which contained about 50% by weight of rhamnolipids in water. Thus, the weight percentages of rhamnolipids in the entire application including in the Examples, Specification, and Claims refer to the weight percentages of the rhamnolipids aqueous solution.

### Hydration Study Results

Examples 8-11 were evaluated against petroleum jelly (positive control) and bare skin for hydration. Subjects cleansed each volar forearm with one 70% isopropyl alcohol wipe. Skin was permitted to air dry for thirty (30) minutes prior to the baseline measurements, with the volar forearms exposed in a room with controlled temperature and humidity conditions. Baseline measurements of each site were taken with the Comcometer 825. Additional measurements were taken at 15 minutes and one hour.

TABLE 2

| | | | | | | | | | | | Ex. | Ex. |
|---|---|---|---|---|---|---|---|---|---|---|---|---|
| Ingredient | Function | Ex. 1 | Ex. 2 | Ex. 3 | Ex. 4 | Ex. 5 | Ex. 6 | Ex. 7 | Ex. 8 | Ex. 9 | 10 | 11 |
| Sodium | Active | 0.350 | 0.350 | 0.300 | 0.300 | 0.300 | 0.300 | 0.350 | 0.450 | 0.450 | 0.250 | 0.450 |
| Hydroxide | Compound | | | | | | | | | | | |
| Tartaric | Active | | | | 0.200 | | | | | | | |
| Acid | Compound | | | | | | | | | | | |
| Lactic Acid | Active | 0.050 | 0.050 | | | 0.050 | 0.050 | 0.050 | 0.050 | 0.050 | 0.050 | 0.050 |
| | Compound | | | | | | | | | | | |
| Trisodium | Active | 0.500 | 0.500 | 0.500 | | 0.500 | 0.500 | 0.500 | 0.500 | 0.500 | 0.500 | 0.500 |
| Ethylenediamine | Compound | | | | | | | | | | | |
| Disuccinate | | | | | | | | | | | | |
| Sodium | Active | 0.250 | 0.250 | 0.250 | 0.250 | 0.250 | 0.250 | 0.250 | 0.250 | 0.250 | 0.250 | 0.250 |
| Lauroyl | Compound | | | | | | | | | | | |
| Lactylate | | | | | | | | | | | | |
| (and) | | | | | | | | | | | | |
| Mixture of | | | | | | | | | | | | |
| Ceramides | | | | | | | | | | | | |
| (and) | | | | | | | | | | | | |
| Phytosphingosine | | | | | | | | | | | | |
| (and) | | | | | | | | | | | | |
| Cholesterol | | | | | | | | | | | | |
| (and) | | | | | | | | | | | | |
| Xanthan | | | | | | | | | | | | |
| Gum (and) | | | | | | | | | | | | |
| Carbomer | | | | | | | | | | | | |
| Cetyl | Fatty | 2.500 | 2.500 | 2.500 | 2.500 | 2.500 | 2.500 | 2.500 | 2.500 | 2.500 | 2.500 | 2.500 |
| Alcohol | Compound | | | | | | | | | | | |
| Kaolin | Filler | 15.0 | 15.0 | 15.0 | 15.0 | 15.0 | 15.0 | 15.0 | 10.0 | 15.0 | 15.0 | 15.0 |
| Carrageenan | Polymer | 1.0 | 1.0 | 1.0 | 1.0 | 1.0 | 1.0 | 1.0 | 0.8 | 1.0 | 1.0 | 1.0 |
| Chitosan | Polymer | 0.10 | 0.10 | 0.10 | 0.10 | 0.10 | 0.10 | 0.10 | 0.10 | 0.10 | 0.10 | 0.10 |
| Salicylic | Active | 2.0 | 2.0 | 2.0 | 2.0 | 2.0 | 2.0 | 2.0 | 2.0 | 2.0 | 2.0 | 2.0 |
| Acid | | | | | | | | | | | | |
| Phenoxyethanol | Preservative | 0.30 | | | | | | 0.30 | | | | |
| Water | Solvent | 55.95 | 51.25 | 56.35 | 56.65 | 51.30 | 56.30 | 55.95 | 23.35 | 48.15 | 48.35 | 28.15 |
| Glycerin | Solvent | 2.0 | 2.0 | 2.0 | 2.0 | 2.0 | 2.0 | 2.0 | 40.0 | 10.0 | 10.0 | 30.0 |
| Glyceryl | Surfactant | 5.0 | 5.0 | 5.0 | 5.0 | 5.0 | 5.0 | 5.0 | 5.0 | 5.0 | 5.0 | 5.0 |
| Stearate SE | | | | | | | | | | | | |
| Sodium | Surfactant | | 10.0 | | | 10.0 | 10.0 | | 10.0 | 10.0 | 10.0 | 10.0 |
| Cocoyl | | | | | | | | | | | | |
| Isethionate | | | | | | | | | | | | |
| Sodium | Surfactant | 10.0 | 5.0 | 10.0 | 10.0 | 5.0 | | 10.0 | | | | |
| Methyl | | | | | | | | | | | | |
| Cocoyl | | | | | | | | | | | | |
| Taurate | | | | | | | | | | | | |
| Rhamnolipids | Surfactant | 3.0 | 3.0 | 3.0 | 3.0 | 3.0 | 3.0 | 3.0 | 3.0 | 3.0 | 3.0 | 3.0 |
| Niacinamide | Active | 2.0 | 2.0 | 2.0 | 2.0 | 2.0 | 2.0 | 2.0 | 2.0 | 2.0 | 2.0 | 2.0 |

The results of the hydration study are provided in Table 3, with the values indicating the level of hydration at baseline, 15 minutes, and 1 hour. As shown in Table 3, Examples 8 through 11 were effective for hydrating the skin at 15 minutes and one-hour intervals, compared to the baseline as well as evaluated against petroleum jelly (positive control) and bare skin.

TABLE 3

| Example | Baseline | 15 min | 1 hr |
|---|---|---|---|
| Petroleum Jelly | 31.47 ± 4.60 | 37.00 ± 9.34 | 39.36 ± 8.06 |
| Bare (Wet) | 31.92 ± 4.82 | 30.37 ± 7.10 | 30.88 ± 7.09 |
| Ex. 8 | 31.54 ± 5.40 | 45.47 ± 9.30 | 45.71 ± 10.23 |
| Ex. 10 | 30.12 ± 5.36 | 32.28 ± 7.64 | 32.83 ± 7.70 |
| Ex. 9 | 29.16 ± 4.94 | 36.07 ± 8.14 | 35.38 ± 8.35 |
| Ex. 11 | 30.28 ± 4.98 | 37.58 ± 7.42 | 38.37 ± 8.78 |

### Examples 12 to 19 of Present Invention

Compositions of Examples 12 to 19 were prepared by mixing the raw materials as listed in Table 4 with a magnetic stirrer. The numerical values for the amounts of the raw materials shown in Table 4 are all based on “% by weight.” Note that rhamnolipids were obtained from EVONIK (trade name: RHEANCE One, INCI name: glycolipids), which contained about 50% by weight of rhamnolipids in water. Thus, the weight percentages of rhamnolipids in the entire application including in the examples, specification, and claims refer to the weight percentages of the rhamnolipids aqueous solution.

TABLE 4

| INCI name | Cosmetic type | Ex 12 | Ex 13 | Ex 14 | Ex 15 | Ex 16 | Ex 17 | Ex 18 | Ex 19 |
|---|---|---|---|---|---|---|---|---|---|
| sodium hydroxide | active | 0.45 | 0.40 | 0.45 | 0.35 | 0.30 | 0.30 | 0.45 | 0.45 |
| | compound | | | | | | | | |
| niacinamide | active | 2.00 | 2.00 | 2.00 | 2.00 | 2.00 | 2.00 | 2.00 | 2.00 |
| | compound | | | | | | | | |
| glyceryl stearatese | surfactant | 5.00 | 5.00 | 5.00 | 5.00 | 5.00 | 5.00 | 5.00 | 5.00 |
| carrageenan | polymer | 1.00 | 1.00 | 1.00 | 1.00 | 1.00 | 1.00 | 0.80 | 1.00 |
| salicylic acid | active | 2.00 | 2.00 | 2.00 | 2.00 | 2.00 | 2.00 | 2.00 | 2.00 |
| | compound | | | | | | | | |
| phenoxyethanol | preservative | | | | 0.30 | | | | |
| water | solvent | 28.15 | 47.70 | 48.15 | 55.95 | 51.30 | 56.30 | 23.35 | 38.15 |
| glycerin | solvent | 30.00 | 10.00 | 10.00 | 2.00 | 2.00 | 2.00 | 40.00 | 20.00 |
| lactic acid | active | 0.05 | 0.05 | 0.05 | 0.05 | 0.05 | 0.05 | 0.05 | 0.05 |
| | compound | | | | | | | | |
| cetyl alcohol | fatty compound | 2.50 | 2.50 | 2.50 | 2.50 | 2.50 | 2.50 | 2.50 | 2.50 |
| kaolin | filler | 15.00 | 15.00 | 15.00 | 15.00 | 15.00 | 15.00 | 10.00 | 15.00 |
| sodium cocoyl | surfactant | 10.00 | 10.00 | 10.00 | | 10.00 | 10.00 | 10.00 | 10.00 |
| isethionate | | | | | | | | | |
| trisodium | active | 0.50 | 1.00 | 0.50 | 0.50 | 0.50 | 0.50 | 0.50 | 0.50 |
| ethylenediamine | compound | | | | | | | | |
| disuccinate | | | | | | | | | |
| sodium lauroyl | active | 0.25 | 0.25 | 0.25 | 0.25 | 0.25 | 0.25 | 0.25 | 0.25 |
| lactylate (and) | compound | | | | | | | | |
| ceramide np (and) | | | | | | | | | |
| ceramide ap (and) | | | | | | | | | |
| phytosphingosine | | | | | | | | | |
| (and) cholesterol | | | | | | | | | |
| (and) xanthan gum | | | | | | | | | |
| (and) carbomer | | | | | | | | | |
| (and) ceramide | | | | | | | | | |
| eop | | | | | | | | | |
| glycolipids | surfactant | 3.00 | 3.00 | 3.00 | 3.00 | 3.00 | 3.00 | 3.00 | 3.00 |
| chitosan | polymer | 0.10 | 0.10 | 0.10 | 0.10 | 0.10 | 0.10 | 0.10 | 0.10 |
| sodium methyl | surfactant | | | | 10.00 | 5.00 | | | |
| cocoyl taurate | | | | | | | | | |

### Sebum Control Study Results

Sebum control studies were conducted according to the following procedure:
1. Wash face with water & acclimatization in standard condition for 30 mins
2. TO baseline measurement on Lightcam
3. Product application as per randomization using standard application gesture
4. Subjects to wait in standard condition for 10 mins
5. Timm measurement on Lightcam
6. Subjects to wait in standard conditions for next 4 hrs for subsequent instrumental measurements

The sebum control study results shown in Table 5 indicates that Examples 12 and 13 had significant reduction of sebum, which outperformed some of the best benchmark products on the markets.

TABLE 5

| | Change in gloss shine |
|---|---|
| Ex 13 | −2.47 |
| Ex 12 | −2.48 |

### Cleansability Study Results

Cleansability studies were carried out according to the procedure illustrated below. Briefly, first apply the sebollution and take a picture for T0, then dilute the cleanser and apply it on sebollution, and finally massage, take another picture at T1.

The calculation for cleansability is also illustrated below. The results are shown in Table 6.

As shown in Table 6, Examples 17, 18, 14, and 12 achieved good cleansability, comparable to the cleansability of benchmark cleansing products on the markets.

$$\begin{matrix}Sebollution \\ application\end{matrix} \begin{matrix}\begin{matrix}Pre - clean with 730327 \; 27 , \\ 2 μ g / {cm}^{2} , Water : product \\ = 2 : 1 ( weight ) , 10 circles \\ mix and messages\end{matrix} \\ \\ \begin{matrix}Wipe by wet cotton , \\ 2 times\end{matrix}\end{matrix} \begin{matrix}Pictured , \\ T 0\end{matrix} \begin{matrix}\begin{matrix}Washing , 2 μ g / cm 2 , \\ Water : product = 2 : 1 \\ ( weight ) , mix and \\ massage 30 circles\end{matrix} \\ \\ \begin{matrix}Wipe by wet cotton , \\ 2 times\end{matrix}\end{matrix} \begin{matrix}Pictured , \\ T 1\end{matrix}$$

$$\begin{matrix}Cleansability = \frac{{De}_{sample cleaned} - {De}_{black reference}}{{De}_{clean reference} - {De}_{black reference}} \\ {D}_{e} = \sqrt{{D}_{l}^{2} + {D}_{a}^{2} + {D}_{b}^{2}}\end{matrix}$$

*De: color difference in a rectangular coordinate system
*Black reference: bioskin with sebollution applied following pore deep clean protocol
*Clean reference: clean bioskin w/o any sebollution

TABLE 6

| | % Cleansability |
|---|---|
| Ex 17 | 67 |
| Ex 18 | 72 |
| Ex 14 | 88 |
| Ex 12 | 85 |

### C. Acne Study Results

The C. acne studies were performed according to the Microbiology department standard procedure I_M_ANA_006_01 “Activity of formulae against microorganisms of cosmetic interest”.

In summary, in a suitable culture medium, we evaluated the biocidal effect on the strain of interest of formulae at 10% after 2, 6 and 24 hours of contact time, in comparison to a control (medium without formula). The reduction in the number of viable bacteria is measured over time. This assay is a “Time Kill” test.

The C. acne results for log reduction compared to the control are shown in Table 7. The C. acne study results for Examples 17, 18, 14, and 12 indicate satisfactory level of reduction.

Note that Examples 17, 18, 14, and 12 had a pH around 5.5. It was further observed that the pH of the samples had significant effect on C. acne reduction. The C. acne results for log reduction with effect of pH are shown in Table 8. A lower pH of 5.5 clearly improved the C. acne reduction compared to pH 7.2.

TABLE 7

| | | 2 hrs. | 6 hrs. | 24 hrs. |
|---|---|---|---|---|
| Ex 17 | Acne Paste Cleanser 2% | 0.6 | 1 | 2.3 |
| | Glycerin | | | |
| Ex 18 | Acne Paste Cleanser 40% | 0.4 | 0.8 | 2 |
| | Glycerin | | | |
| Ex 14 | Acne Paste Cleanser 10% | 0.6 | 0.9 | 2 |
| | Glycerin | | | |
| Ex 12 | Acne Paste Cleanser 30% | 0.6 | 1 | 2 |
| | Glycerin | | | |

TABLE 8

| | | | Log reduction compared | | |
|---|---|---|---|---|---|
| | | | to the Control | | |
| | | | 2 | 6 | 24 |
| | | | hrs. | hrs. | hrs. |
| PH | Ex 16 | Acne Paste Cleanser 2% | 1.9 | 3.9 | 5 |
| 5.5 | | Glycerin + 10% Sodium Cocoyl | | | |
| | | Isethionate | | | |
| | Ex 15 | Acne Paste Cleanser 2% Glycerin | 1.5 | 1.4 | 4.1 |
| pH | Ex 16 | Acne Paste Cleanser 2% | 0 | 0.2 | 0.6 |
| 7.2 | | Glycerin + 10% Sodium Cocoyl | | | |
| | | Isethionate | | | |
| | Ex 15 | Acne Paste Cleanser 2% Glycerin | 0 | 0 | 0 |

### Three-In-One Applications of The Acne Paste Formulas

The inventors found that when substituting the rheology agent of the inventive composition, xanthan gum and hydroxypropyl guar resulted in a slimy texture, hydroxypropyl starch phosphate resulted in texture that was not thickened, ammonium polyacryloyldimethyl taurate and polyacrylate crosspolymer-6 resulted in texture a that was not smooth, and acrylates/C10-C30 alkyl acrylate crosspolymer and carbomer resulted in a texture that was not smooth and the composition required neutralization of pH for suitable use.

Referring to table 4, Examples 12, 14 and 18 were developed to be 3-in-1 formulas for a daily skin clearing cleanser when mixed with water, an overnight drying spot treatment in its original form, and a deeply purifying mask when layered on damped face.

Examples 12, 14 and 18 contained optimal concentrations of kaolin, solid mild surfactant (sodium cocoyl isethionate) and glycerin for fast-drying, sebum control, and hydration. They also contained effective amounts of salicylic acid, rhamnolipids, and niacinamide for anti-bacterial and anti-inflammatory treatments; and sufficient amount of chitosan for enhanced salicylic acid deposition.

Moreover, Examples 12, 14 and 18 had a unique wasabi-like paste texture suitable for the 3-in-1 applications, which also require many optimized physical attributes such as, but not limited to, spreadability, drying rate, miscibility with water, foaming, rinsibility, and appearance etc. The attributes were rated as acceptable, fair, or good. As shown is Table 9, Examples 12 and 14 had better physical attributes in general than Example 18.

TABLE 9

| | Cleanser | | | | | | Spot Treatment | | |
|---|---|---|---|---|---|---|---|---|---|
| | | Quickness | | | | | | Stay | |
| | Miscibility | of | Quickness | Mask | | | Quick | in | |
| | w/ water | Foaming | of Rinsing | Spreadability | Coverage | Rinsibility | Drying | Place | Clear |
| EX | acceptable | acceptable | fair | Good | fair | good | acceptable | fair | good |
| #18 | | | | excellent | | | | | |
| EX | good | fair | good | Fair to good | fair | good | good | good | fair |
| #14 | | | | | | | | | |
| EX | fair | good | good | Fair to good | fair | good | acceptable | fair | good |
| #12 | | | | | | | | | |

1. A cosmetic composition comprising at least one glycolipid selected from the group consisting of rhamnolipids, sophorolipids, glucolipids, trehalolipids, cellobiose lipids and mixtures thereof, at least one salicylic acid or derivative, at least on cationic polymer comprising a polysaccharide derived from β-linked D-glucosamine and N-acetyl-D-glucosamine, at least one clay, cosmetically acceptable solvents, at least one surfactant, and at least one thickener/rheology modifier.

2. The cosmetic composition according to claim 1 wherein the at least one glycolipid comprises a rhamnolipid, the at least one salicylic acid or derivative comprises salicylic acid, the at least on cationic polymer comprises chitosan, at least one clay comprises kaolin, the cosmetically acceptable solvents comprise at least water and glycerin, the at least one surfactant that is a solid surfactant that comprises sodium cocoyl isethionate, at least one additional skin active that comprises niacinamide, and at least one thickener/rheology modifier that comprises carrageenan.

3. The cosmetic composition according to claim 2, wherein the at least one rhamnolipid is present from about 2% to about 5%, salicylic acid is present from about 1% to about 3%, kaolin present from about 10% to about 20%, sodium cocoyl isethionate is present from about 5% to about 15%, glycerin present from about 10% to about 40%, niacinamide is present at about 2%, carrageenan is present from about from about 0.5% to about 2%, all amounts by weight, relative to the total weight of the composition, and wherein the pH of the composition is from about 3.5 to about 6.5.

4. The cosmetic composition according to claim 2 wherein the composition has a unique paste texture that is sufficiently thick to not run or slip off the skin when applied prior to rinsing, is not slimy, and is smooth without granulation or graininess, is water miscible and glides on when applied with shear stress, with low to high foaming and which dries clear.

5. A method for treating, hydrating, and/or cleansing acne-prone skin, comprising applying the composition of claim 1 onto the acne-prone skin.

6. A cosmetic composition comprising:
(a) at least one glycolipid;
(b) at least one salicylic acid or its derivatives of formula (I): 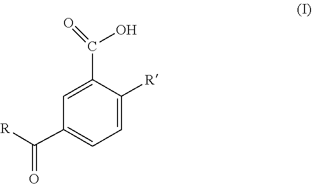
in which:
the radical R denotes a linear, branched or cyclic, saturated aliphatic chain containing from 2 to 22 carbon atoms; an unsaturated chain containing from 2 to 22 carbon atoms containing one or more double bonds that may be conjugated; an aromatic nucleus linked to the carbonyl radical directly or via saturated or unsaturated aliphatic chains containing from 2 to 7 carbon atoms; said groups possibly being substituted with one or more substituents, which may be identical or different, chosen from halogen atoms, a trifluoromethyl group, hydroxyl groups in free form or esterified with an acid containing from 1 to 6 carbon atoms, or carboxyl groups in free form or esterified with a lower alcohol containing from 1 to 6 carbon atoms;
R′ is a hydroxyl group or an ester group of formula: 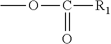
in which R1 denotes a linear or branched, saturated or unsaturated aliphatic chain containing 1 to 18 carbon atoms; and also salts thereof derived from an inorganic or organic base;
(c) at least one cationic polymer selected from a polysaccharide derived from β-linked D-glucosamine or N-acetyl-D-glucosamine;
(d) at least one clay present from about 5% to about 25% by weight, relative to the total weight of the composition;
(e) cosmetically acceptable solvents comprising water, and comprising glycerin present from about 2% to about 40% by weight, relative to the total weight of the composition; and
(f) at least one surfactant present from about 1% to about 20% by weight, relative to the total weight of the composition,
wherein the pH of the composition is from about 3.5 to about 6.5, and
wherein the composition has a wasabi-like paste texture.

7. The composition according to claim 6, wherein the at least one glycolipid is selected from the group consisting of rhamnolipids, sophorolipids, glucolipids, trehalolipids, cellobiose lipids, and mixtures thereof, and wherein the amount of the at least one glycolipid is from 0.1% by weight to 10% by weight, relative to the total weight of the composition.

8. The composition according to claim 7, wherein the at least one glycolipid is rhamnolipids.

9. The composition according to claim 8, wherein the amount of the rhamnolipids is from 2% by weight to 5% by weight, relative to the total weight of the composition.

10. The composition according to claim 6, wherein the at least one salicylic acid or its derivatives is salicylic acid, and wherein the amount of the at least one salicylic acid or its derivatives is from 0.1% by weight to 5% by weight, relative to the total weight of the composition.

11. The composition according to claim 6, wherein the cationic polymer is chitosan.

12. A cosmetic composition comprising:
(a) at least one rhamnolipid present from 2% by weight to 5% by weight, relative to the total weight of the composition;
(b) at least one salicylic acid or its derivatives or its derivatives of formula (I): 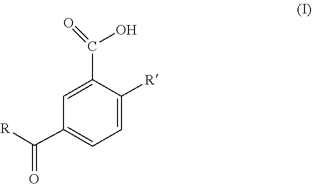
in which:
the radical R denotes a linear, branched or cyclic, saturated aliphatic chain containing from 2 to 22 carbon atoms; an unsaturated chain containing from 2 to 22 carbon atoms containing one or more double bonds that may be conjugated; an aromatic nucleus linked to the carbonyl radical directly or via saturated or unsaturated aliphatic chains containing from 2 to 7 carbon atoms; said groups possibly being substituted with one or more substituents, which may be identical or different, chosen from halogen atoms, a trifluoromethyl group, hydroxyl groups in free form or esterified with an acid containing from 1 to 6 carbon atoms, or carboxyl groups in free form or esterified with a lower alcohol containing from 1 to 6 carbon atoms;
R′ is a hydroxyl group or an ester group of formula: 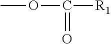
in which R1 denotes a linear or branched, saturated or unsaturated aliphatic chain containing 1 to 18 carbon atoms; and also salts thereof derived from an inorganic or organic base;
the at least one salicylic acid or its derivatives or its derivatives of formula (I) being present from about 1% by weight to about 3% by weight, relative to the total weight of the composition;
(c) at least one cationic polymer comprising chitosan;
(d) at least one clay present from about 5% to about 25% by weight, relative to the total weight of the composition;
(e) cosmetically acceptable solvents comprising water, and comprising glycerin present from about 10% to about 40% by weight, relative to the total weight of the composition; and
(f) at least one surfactant present from about 1% to about 20% by weight, relative to the total weight of the composition,
wherein the pH of the composition is from about 3.5 to about 6.5,
wherein the composition has a wasabi-like paste texture, and
wherein the composition is a 3-in-1 paste formula base for acne-prone skin as a skin cleanser, as a spot treatment, and as a mask.

13. The composition of claim 12, wherein the at least one surfactant includes sodium cocoyl isethionate present from about 5% to about 15% by weight, relative to the total weight of the composition.

14. The composition of claim 12, further comprising niacinamide present at about 2% by weight, relative to the total weight of the composition.

15. The composition of claim 12, wherein the at least one clay includes kaolin present from about 10% to about 20% by weight, relative to the total weight of the composition.

16. The composition of claim 12, wherein the at least one clay includes kaolin present from about 10% to about 20% by weight, relative to the total weight of the composition and the at least one surfactant includes sodium cocoyl isethionate present from about 5% to about 15% by weight, relative to the total weight of the composition.

17. The composition of claim 16, wherein the kaolin is present at about 15% by weight, relative to the total weight of the composition and the sodium cocoyl isethionate is present at about 10% by weight, relative to the total weight of the composition.

18. The composition according to claim 12, wherein the composition is one of a daily clearing skin cleanser to be mixed with water, an overnight drying spot treatment in its original form, a mask to be layered on damped face, or a combination thereof.

19. A cosmetic composition comprising at least one glycolipid selected from the group consisting of rhamnolipids, sophorolipids, glucolipids, trehalolipids, cellobiose lipids and mixtures thereof, at least one salicylic acid or derivative, at least on cationic polymer comprising a polysaccharide derived from β-linked D-glucosamine and N-acetyl-D-glucosamine, at least one clay, cosmetically acceptable solvents, at least one surfactant, and at least one thickener/rheology modifier, wherein the at least one glycolipid comprises at least one rhamnolipid present from about 2% to about 5%, the composition comprises salicylic acid present from about 1% to 3%, the at least one clay comprises kaolin present at about 15%, the at least one surfactant comprises sodium cocoyl isethionate present at about 10%, the cosmetically acceptable solvents comprise glycerin present from about 10% to about 40%, the composition comprises at least one additional skin active that comprises niacinamide present at about 2%, the at least one thickener/rheology modifier comprises carrageenan present from about from about 0.5% to about 2%, all amounts by weight, relative to the total weight of the composition, wherein the pH of the composition is from about 3.5 to about 6.5, and wherein the composition has a unique paste texture that is sufficiently thick to not run or slip off the skin when applied prior to rinsing, is not slimy, and is smooth without granulation or graininess, is water miscible and glides on when applied with shear stress, with low to high foaming and which dries clear, wherein the kaolin is present at about 15% by weight, relative to the total weight of the composition and the sodium cocoyl isethionate is present at about 10% by weight, relative to the total weight of the composition.
